(19) United States
(12) Patent Application Publication
(10) Pub. No.: US 2008/0051619 A1
(43) Pub. Date: Feb. 28, 2008

(54) Olefin-Separation Process

(76) Inventors: Santi Kulprathipanja, Inverness, IL (US); Darryl M. Johnson, Broadview, IL (US); Stephen W. Sohn, Arlington Heights, IL (US)

Correspondence Address: HONEYWELL INTELLECTUAL PROPERTY INC;PATENT SERVICES, 101 COLUMBIA DRIVE, P O BOX 2245 MAIL STOP AB/2B, MORRISTOWN, NJ, 07962, US

(21) Appl. No.: 11/467,383

(22) Filed: Aug. 25, 2006

(51) Int. Cl. C07C 7/12

(52) U.S. Cl. 585/821

(57) ABSTRACT

This invention is drawn to a process for recovering detergent-range olefins from a feed stream by adsorption. The adsorbent and desorbent are selected to enable olefins with a range of carbon numbers to be recovered simultaneously in light of differing adsorbent retention characteristics.

### FIELD OF THE INVENTION

The present invention relates to the separation of hydrocarbon species. More specifically, the invention embodies a process for the adsorptive separation of olefinic from paraffinic hydrocarbons using a specific type of hydrocarbon as desorbent.

### BACKGROUND OF THE INVENTION

Olefinic hydrocarbons are widely useful petrochemical intermediates. Important chemical products are formed by olefin polymerization, oligomerization and alkylation with other chemical species. It is often necessary for the olefins to be relatively high in purity for effective process reactions or to minimize byproduct formation. Most frequently, it is necessary or at least desirable to separate the olefins from nonolefinic hydrocarbons such as paraffins. Often it is desirable to separate one particular type of olefin such as a normal olefin or alpha olefin from a mixture comprising other types of olefins such as branched-chain olefins.

In an admixture of a desired olefin with a chemical species of different relative volatility, the olefin may be recovered from the admixture by straightforward fractional distillation. If the olefin is present in a mixture containing one or more different hydrocarbons having similar volatilities, however, separation may be difficult or impossible by distillation. One common example of this occurs when the olefins are produced by the dehydrogenation of a paraffin or a mixture of paraffins. As the dehydrogenation reaction will not proceed to completion due to equilibrium constraints, the dehydrogenation product is a homologous mixture of paraffins and olefins having very similar boiling points. Fractional distillation usually is impractical in this instance, and adsorptive separation utilizing an adsorbent which is selective for olefins often is the most effective separation method.

It is known in the art that adsorptive separation is an effective method to separate linear olefinic hydrocarbons from a feed mixture comprising the linear olefinic hydrocarbons and another class of hydrocarbons having a similar volatility such as paraffins or nonlinear olefins of the same general molecular weight. This process is described in a paper entitled Olex: A Process for Producing High Purity Olefins presented by J. A. Johnson, S. Raghuram and P. R. Pujado at the August 1987 Summer national meeting of the American Institute of Chemical Engineers in Minneapolis, Minn. This paper describes a simulated-moving-bed (SMB) countercurrent adsorptive separation process for the separation of light straight-chain olefins from similar paraffins. A similar but more detailed description of SMB for the separation of linear olefins is provided in U.S. Pat. No. 3,510,423 issued to R. W. Neuzil et al.

U.S. Pat. No. 5,276,246 issued to Beth McCulloch et al. describes a process for the adsorptive separation of C₅ to C₈ normal olefins from a mixture of normal olefins and branched-chain olefins using a low-acidity silica molecular sieve such as a silicalite or ZSM molecular sieve with a desorbent consisting essentially of alkyl-substituted cycloparaffins.

U.S. Pat. No. 5,300,715 issued to B. V. Vora describes an overall process for the conversion of paraffins to olefins. The process includes dehydrogenation of the paraffins and adsorptive separation of the olefins from a paraffin/olefin mixture recovered from the effluent of the dehydrogenation zone. The patent describes a zone used to selectively remove aromatic hydrocarbons from the paraffin/olefin mixture to prevent the aromatic hydrocarbons from deactivating a molecular sieve used in the adsorptive separation of the paraffin/olefin mixture and to aid the performance of the dehydrogenation.

U.S. Pat. No. 6,106,702 discloses an adsorptive separation process for separating olefins from paraffins wherein a guard bed is employed to remove aromatic hydrocarbon contaminants from the feed stream. An existing internal desorbent stream is used as the flush for the guard bed and is regenerated in the raffinate column of the process.

### SUMMARY OF THE INVENTION

A broad embodiment of the present invention is an adsorptive separation process for the separation of detergent-range olefinic hydrocarbons from a feed stream comprising one or more olefinic hydrocarbons and other hydrocarbon species, comprising contacting the feed stream with a bed of adsorbent under conditions which cause the selective retention of the detergent-range olefinic hydrocarbons on the adsorbent and recovering the retained detergent-range olefinic hydrocarbons from the adsorbent by contacting the adsorbent with a desorbent comprising one or more naphthenic hydrocarbons.

A more specific embodiment is an adsorptive separation process for the separation of detergent-range linear olefinic hydrocarbons from a feed stream comprising one or more olefinic hydrocarbons and other hydrocarbon species, comprising contacting the feed stream with a bed of adsorbent under conditions which cause the selective retention of the detergent-range linear olefinic hydrocarbons on the adsorbent and recovering the retained linear detergent-range olefinic hydrocarbons from the adsorbent by contacting the adsorbent with a desorbent comprising one or more naphthenic hydrocarbons.

A yet more specific embodiment is a simulated-moving-bed adsorptive separation process for the separation of detergent-range olefinic hydrocarbons from a feed stream comprising one or more olefinic hydrocarbons and other hydrocarbon species, comprising contacting the feed stream with a bed of adsorbent comprising Type X zeolite under conditions which cause the selective retention of the detergent-range olefinic hydrocarbons on the adsorbent and recovering the retained detergent-range olefinic hydrocarbons from the adsorbent by contacting the adsorbent with a desorbent comprising one or more naphthenic hydrocarbons.

### BRIEF SUMMARY OF THE DRAWINGS

FIG. 1 illustrates the significance of measuring net retention value (NRV) in comparing desorbents.

FIG. 2 compares pulse-test results for desorbent B and a cyclohexane desorbent on a feed containing nC₁₄= and nC₁₄.

FIG. 3 compares pulse-test results for desorbent B and a cyclohexane desorbent on a feed containing nC₁₆= and nC₁₆.

### DETAILED DESCRIPTION AND PREFERRED EMBODIMENTS

An olefin-containing feed stream to the present process may be derived from any of a variety of sources containing linear or branched-chain olefins having appropriate detergent-range carbon chain lengths. A typical feed stream is produced by the dehydrogenation of normal paraffins derived by extraction from a kerosene-range petroleum fraction. Another potential source is an olefinic stream derived from Fischer-Tropsch synthesis. The feed source is not limiting of the invention.

“Detergent-range olefinic hydrocarbons” comprising the product of the present invention contain one or more olefins within the range of C₉ to C₂₀, i.e., consist essentially of olefinic hydrocarbons having between 9 and 20 carbons in each molecule. More typically, the carbon-number range is between 9 and 16, with 10 to 14 often being preferred and a range of 11 to 13 being appropriate for specific detergent properties. The present invention is particularly advantageous relative to the known art, when the product has a wider carbon range of at least three carbon numbers, preferably four or more, and especially when the range of carbon numbers is at least five. The content of C₈ and lighter olefins generally is less than about 1.0 wt.-%, typically less than about 0.5 wt.-%, and preferably less than 0.1 wt.-%.

A preferred use of the olefins is in the production of detergent ingredients or precursor compounds such as alkylbenzenes, which may then be converted to a linear alkylsulfonate (LAS) by sulfonation with sulfur trioxide or sulfuric acid followed by neutralization. The product olefins can also be used in the production of other detergent precursors or ingredients including ethoxylates and linear alcohol sulfates by known reactions. If branched olefinic hydrocarbons are produced, these may be converted to cleaning product ingredients by alkylation with toluene or phenol followed by alkoxylation or sulfonation, or by hydroformulation followed by a secondary step such as alkoxylation, sulfation, phosphation, oxidation or a combination of these steps.

The nonrecovered hydrocarbons in the feed stream may be a different type of olefin or paraffins or a mixture of olefins and paraffins; other hydrocarbon species, e.g., naphthenes and aromatics, also may be present. The process may therefore be specific to the recovery of normal olefin(s) from a mixture comprising isoolefins and/or paraffins.

An adsorptive separation process basically comprises an adsorption step performed in which the adsorbent is brought into contact with the olefin-containing feed at adsorption conditions and a desorption step in which selectively adsorbed olefins are removed from the adsorbent at desorption conditions. Adsorptive separation can be performed using a variety of different techniques such as a swing-bed operation using two or more fixed beds with adsorption and regeneration steps cycling between them, moving bed operation in which the adsorbent is transported between adsorption and desorption zones, and simulated-moving-bed (SMB) operation such as described in U.S. Pat. Nos. 2,985,589; 3,510,423; 3,720,604; 3,723,302 and 3,755,153. The preferred system for the present separation is a countercurrent simulated-moving-bed (SMB) system. Cyclic advancement of the input and output streams in an SMB operation can be accomplished by a manifolding system or by rotary disc valves, which are also known, e.g., shown in U.S. Pat. Nos. 3,040,777 and 3,422,848. These patents are incorporated herein for their background teaching as to SMB separation techniques, nomenclature and for their description of adsorbents useful for adsorptive separations. Notwithstanding the description of the preferred system, the manner in which the adsorbent is contacted with the feed stream is not a limiting factor in the subject invention.

Simulated-moving-bed adsorptive separation units typically simulate countercurrent movement of the adsorbent and the feed stream, though simulated co-current movement of the adsorbent and feed stream is also known. A thorough explanation of SMB processes is given in the Adsorption, Liquid Separation section of the Kirk-Othmer Encyclopedia of Chemical Technology.

Simulated-moving-bed processes typically include at least three or four separate steps which are performed sequentially in separate zones within a mass of adsorbent retained in one or more vertical cylindrical adsorption chambers. Each of these zones normally is formed from a plurality of beds of adsorbent, sometimes referred to as sub-beds, with the number of beds per zone ranging from 2 or 3 up to 8-10. The most widely practiced commercial process units typically contain about 24 beds. All of the beds are contained in one or more vertical vessels referred to herein collectively as the adsorbent chamber. The beds are structurally separated from one another by a horizontal liquid collection/distribution grid. Each grid is connected to a transfer line defining a transfer point at which process streams such as the feed stream and raffinate and extract streams enter or leave the vertical adsorption chambers.

Various terms used herein are defined as follows. An “extract” is a compound or class of compounds that is more selectively adsorbed by the adsorbent, representing the olefinic hydrocarbon product, while a “raffinate” is a compound or class of compound that is less selectively adsorbed. The term “desorbent” means generally a material capable of and used for desorbing an extract component from the adsorbent. The term “extract stream” means a stream in which the extract, which has been desorbed by a desorbent material, is removed from the adsorbent bed. The term “raffinate stream” means a stream in which a raffinate component is removed from the adsorbent bed after the adsorption of extract compounds

The positions at which the streams involved in the process enter and leave the chambers are slowly shifted from sub-bed to sub-bed along the length of the adsorbent chambers so that the streams enter or leave different sub-beds as the operational cycle progresses. Normally there are at least four streams (feed stream, desorbent, extract and raffinate streams) employed in this procedure, and the location at which the feed stream and desorbent enter the chamber and the extract and raffinate streams leave the chamber are simultaneously shifted in the same direction at set intervals. Each periodic incremental shift in the location of these transfer points delivers or removes liquid from a different sub-bed of adsorbent within the chamber. This shifting could be performed using a dedicated line for each stream at the entrance to each sub-bed. However, this would greatly increase the cost of the process and therefore the lines are typically reused. Only one line is normally employed for each sub-bed, and each bed line carries one of the four process streams at some point in the cycle. This simulation procedure normally also includes the use of a variable flow rate pump which pushes liquid leaving one end of the adsorbent vessel(s) to the other end in a single continuous loop.

The extract stream and the raffinate stream generally are passed to separation means, typically fractional distillation columns, where at least a portion of desorbent is recovered and an extract product and a raffinate product are produced.

The adsorbents employed in the subject process are preferably molecular sieves formed from inorganic oxides such as silica and alumina; that is, aluminosilicates. Such materials include the well known commercially available zeolites such as zeolite Y and zeolite X. The microcrystalline sieve structure provided by many zeolites is important in the selectivity of the adsorbent for the olefinic hydrocarbon. The term molecular sieve is intended to include a broad variety of inorganic oxides which are suitable as guard bed adsorbents and/or as adsorbents for the separation of olefins including the silicalite materials described in the above cited references. Silicalites are very high silica to alumina ratio molecular sieves which are not zeolites due to their lack of ion exchange capacity. Silicalites are described in greater detail in U.S. Pat. Nos. 4,061,724; 4,073,865 and 4,104,294. Another type of inorganic oxide molecular sieve which could be used in the adsorbent is the ZSM type zeolite such as disclosed in U.S. Pat. No. 3,702,886 (ZSM-5), U.S. Pat. No. 3,832,449 (ZSM-12), U.S. Pat. No. 4,016,245 (ZSM-35) and U.S. Pat. No. 4,046,859 (ZSM-38).

The preferred adsorbent for use in the separation zone is an attrition resistant particle of about 20-40 mesh (U.S.) size formed by extrusion or spray drying an admixture of a binder such as clay or alumina and a type X or type Y zeolite. The type X zeolite is described in U.S. Pat. No. 2,822,244 and the type Y zeolite is described in U.S. Pat. No. 3,130,007. The zeolites may be ion exchanged to replace native sodium with one or more other cations selected from the alkali metals, and/or the alkaline-earth metals. Preferred metals include lithium, potassium, calcium, strontium and barium. Combinations of two or more of these metals may be employed. The preferred level of ion-exchange, if any, of these materials is rather low. One highly preferred adsorbent is a sodium form 13× zeolite.

One operational problem related to the adsorptive separation of olefins can be the accumulation of certain compounds, present in the feed stream, on the active sites of the adsorbent. These compounds tend to bind so tightly to the sites that the desorption procedure used for olefin recovery does not remove them. As the deleterious effects grow due to the accumulation of more poison from the feed stream, the capacity of the adsorbent and thus the overall process is decreased. The most common ones encountered in the subject process comprise diolefins and aromatic hydrocarbons. The art has recognized that it is desirable to prevent poisons from deactivating the molecular sieves used to separate olefins as shown by the processes described in U.S. Pat. Nos. 5,276,246; 5,300,715 and 6,106,702, incorporated herein by reference thereto.

A desorbent material for use in a liquid-phase adsorption process must be judiciously selected to satisfy several criteria. First, the desorbent material should displace an extract component from the adsorbent with reasonable mass flow rates without itself being so strongly adsorbed as to unduly prevent an extract component from displacing the desorbent material in a following adsorption cycle. Expressed in terms of the selectivity, it is preferred that the adsorbent be more selective for all of the extract components with respect to a raffinate component than it is for the desorbent material with respect to a raffinate component. Secondly, desorbent materials must be compatible with the particular adsorbent and the particular feed mixture. More specifically, they must not reduce or destroy the capacity of the adsorbent or selectivity of the adsorbent for an extract component with respect to a raffinate component. Additionally, desorbent materials should not chemically react with or cause a chemical reaction of either an extract component or a raffinate component. Both the extract stream and the raffinate stream are typically removed from the adsorbent void volume in admixture with desorbent material and any chemical reaction involving a desorbent material and an extract component or a raffinate component or both would complicate or prevent product recovery. The desorbent should also be easily separated from the extract and raffinate components, as by fractionation. Finally, desorbent materials should be readily available and reasonable in cost.

For use in recovering detergent-range olefinic products according to the present process, the desorbent comprises naphthenic hydrocarbons. It has been observed that these are particularly suitable when recovering a range of olefinic hydrocarbons, wherein the selectivity is a function of the olefin carbon number as well as the hydrocarbon type since net retention volume is similar over a range of carbon numbers. Suitable naphthenic hydrocarbons include one or more alkylcyclopentanes and cyclohexanes in the C₆ to C₈ range which can be separated readily from detergent-range olefinic products by fractionation. The desorbent should have a content of naphthenic hydrocarbons of at least 90 wt.-%. It is preferred that the naphthenic desorbent consists essentially of one or both of methylcyclopentane and cyclohexane, with cyclohexane being especially preferred.

Adsorption conditions in general include a temperature range of from about 20° to about 250° C., with from about 40° to about 150° C. being highly preferred and temperatures from 50° to 100° C. being especially preferred. Adsorption conditions also preferably include a pressure sufficient to maintain the process fluids in liquid phase; which may be from about atmospheric to 4.5 MPa. Desorption conditions generally include the same temperatures and pressure as used for adsorption conditions. Variations within and near to these limits depend on the composition of the adsorbent and the feed.

### EXAMPLES

A “pulse test” procedure was employed to test alternative desorbents with a particular feed mixture and Na-Type X zeolite adsorbent. The basic pulse test apparatus consists of a tubular adsorbent chamber of approximately 70 cc volume having an inlet and outlet at opposite ends of the chamber. The chamber is contained within a temperature control means and pressure control equipment is used to maintain the chamber at a constant predetermined pressure. Quantitative and qualitative analytical equipment such as refractometers, polarimeters and chromatographs can be attached to an outlet line of the chamber and used to detect quantitatively and/or determine qualitatively one or more components in the effluent stream leaving the adsorbent chamber. During a pulse test, the adsorbent is first filled to equilibrium with a particular desorbent material by passing the desorbent material through the adsorbent chamber. A pulse of the feed mixture, sometimes diluted in desorbent, is then injected for a duration of one or more minutes. Desorbent flow is resumed, and the feed components are eluted as in a liquid-solid chromatographic operation.

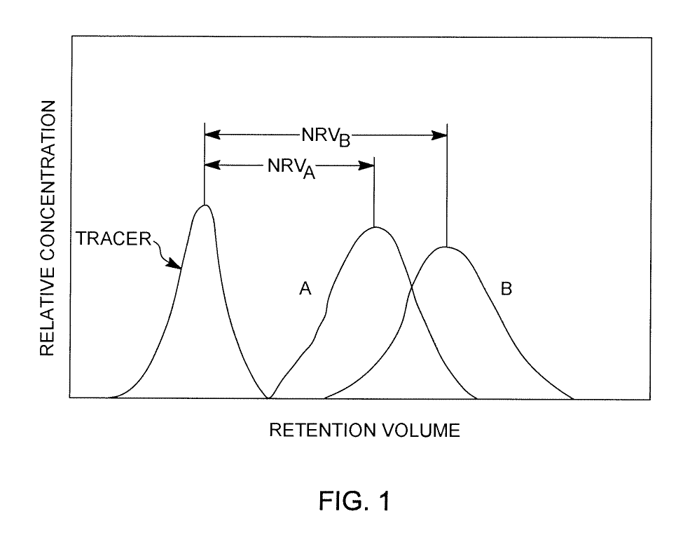

Desorbents were compared by measuring net retention value (“NRV”), the significance of which can be understood by reference to FIG. 1 which illustrates a hypothetical pulse test. The feed to the hypothetical test contains components A and B and a tracer selected to not be absorbed by the system being studied. The peak of the tracer is set as the zero origin on the volume scale, and the peak of each of components A and B are indexed as their respective NRV on the volume scale at the midpoint of the peak. Since NRV is ideally proportional to its distribution coefficient between the adsorbed phase and unadsorbed phase, the selectivity of the 2 components can be calculated by the ratio NRV.

Test results were based on a series of feedstocks comprising 10% normal olefin, 85 wt.-% normal paraffin, and 5 wt.-% n-C₁₈ as a tracer. Each olefin/paraffin pair comprised the same carbon number, e.g. n-nonene was paired with n-nonane. Net retention volume (NRV) was measured for each pair and expressed as NRV olefin/paraffin. The various desorbents tested were:

| | |
|---|---|
| A | 80/20 n-heptane/1-octene |
| B | 60/40 n-hexane/1-hexene |
| MCP | methylcyclopentane |
| MCH | methylcyclohexane |
| CH | cyclohexane |

### Results were as follows as NRV for each pair at 125° C.:

| | Desorbent: | | | | |
|---|---|---|---|---|---|
| | A | B | MCP | MCH | CH |
| nC₉=/nC₉ | 26.09/2.67 | 15.94/1.98 | 23.21/2.7 | 31.51/3.14 | 17.68/2.46 |
| nC₁₀=/nC₁₀ | 17.35/1.62 | 12.0/1.58 | | | 13.62/1.90 |
| nC₁₂=/nC₁₂ | 11.1/0.97 | 6.82/1.18 | | | 7.50/1.0 |
| nC₁₄=/nC₁₄ | 7.16/0.23 | 4.03/0.19 | | | 6.22/0.50 |
| nC₁₆=/nC₁₆ | 5.6/0.03 | 3.59/0.8 | 5.38/0.31 | 10.12/0.58 | 6.63/0.43 |

These results lead to the following conclusions regarding suitable desorbents for recovery of this range of olefins:

Although Desorbent A may be useful for separations involving a single or small range of carbon numbers, it is impractical for a feed having a wide range of carbon numbers such as illustrated here because the NRV of different carbon-number olefins varies too greatly. Desorbent A particularly is not acceptable for the processing of a feed containing a significant concentration of C₉ olefins, because the boiling point is similar to that of the product which renders separation of product from the desorbent impractical.

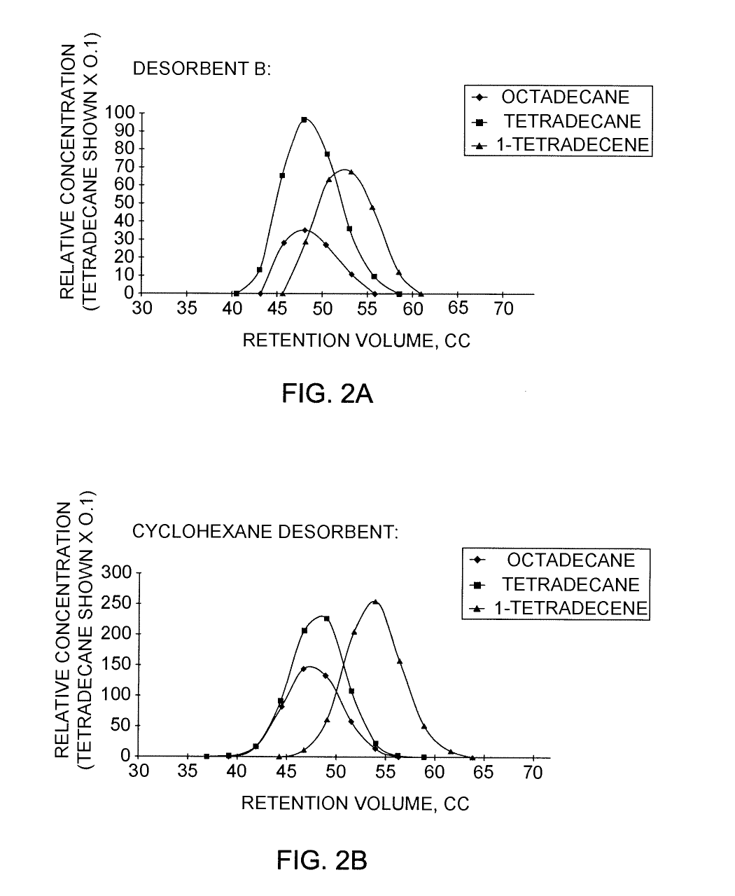

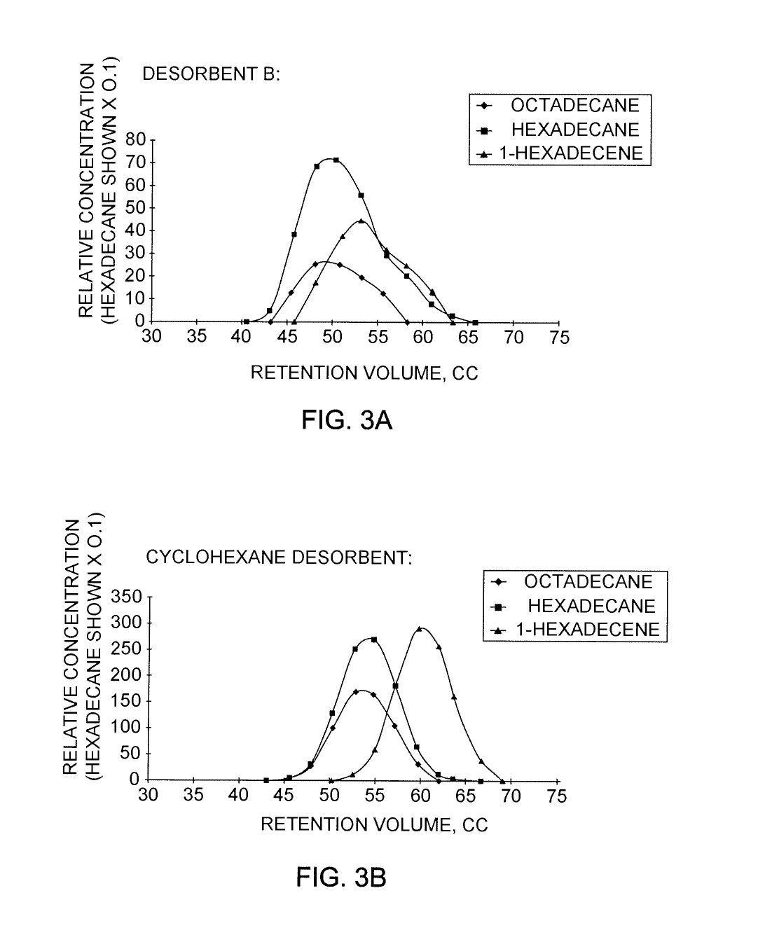

Desorbent B is impractical for feeds containing certain higher carbon numbers even though NRVs may indicate utility. For example, FIG. 2 shows a substantial overlap of the desorption peaks of nC₁₄= and nC₁₄ (the nC₁₄ concentration was divided by 10 to place it on the same scale). FIG. 3 shows an even greater overlap of nC₁₆= and nC₁₆ (the nC₁₆ concentration was divided by 10 to place it on the same scale), indicating that cannot be separated with this desorbent. FIGS. 2 and 3 show comparative desorption peaks showing that a cyclohexane desorbent could achieve separation of the respective olefin and paraffin.

Thus, naphthenes, especially methylcyclopentane, methylcyclohexane and cyclohexane are useful desorbents for the separation of detergent range olefins. Naphthenes provide additional advantages when olefins over a range of carbon numbers are separated together from the feed stream.

We claim:

1. An adsorptive separation process for the separation of detergent-range olefinic hydrocarbons from a feed stream comprising one or more olefinic hydrocarbons and other hydrocarbon species, comprising contacting the feed stream with a bed of adsorbent under conditions which cause the selective retention of the detergent-range olefinic hydrocarbons on the adsorbent and recovering the retained detergent-range olefinic hydrocarbons from the adsorbent by contacting the adsorbent with a desorbent comprising one or more naphthenic hydrocarbons.

2. The process of claim 1 wherein the detergent-range olefinic hydrocarbons comprise olefins within the range of C₉ to C₂₀.

3. The process of claim 2 wherein the detergent-range olefinic hydrocarbons consist essentially of olefins within the range of C₉ to C₂₀.

4. The process of claim 3 wherein the olefinic hydrocarbons have a carbon-number range of at least three.

5. The process of claim 1 wherein the adsorbent comprises a molecular sieve.

6. The process of claim 5 wherein the molecular sieve comprises a Type X zeolite.

7. The process of claim 1 wherein the naphthenic hydrocarbons consists essentially of one or both of cyclohexane and methylcyclopentane.

8. The process of claim 7 wherein the naphthenic hydrocarbons consist essentially of cyclohexane.

9. The process of claim 1 wherein the adsorptive separation process is a simulated-moving-bed adsorptive separation process.

10. An adsorptive separation process for the separation of detergent-range linear olefinic hydrocarbons from a feed stream comprising one or more olefinic hydrocarbons and other hydrocarbon species, comprising contacting the feed stream with a bed of adsorbent under conditions which cause the selective retention of the detergent-range linear olefinic hydrocarbons on the adsorbent and recovering the retained linear detergent-range olefinic hydrocarbons from the adsorbent by contacting the adsorbent with a desorbent comprising one or more naphthenic hydrocarbons.

11. The process of claim 10 wherein the detergent-range linear olefinic hydrocarbons comprise linear olefins within the range of C₉ to C₂₀.

12. The process of claim 11 wherein the detergent-range linear olefinic hydrocarbons consist essentially of linear olefins within the range of C₉ to C₂₀.

13. The process of claim 10 wherein the adsorbent comprises a molecular sieve.

14. The process of claim 13 wherein the molecular sieve comprises a Type X zeolite.

15. The process of claim 10 wherein the naphthenic hydrocarbons consists essentially of one or both of cyclohexane and methylcyclopentane.

16. The process of claim 10 wherein the adsorptive separation process is a simulated-moving-bed adsorptive separation process.

17. A simulated-moving-bed adsorptive separation process for the separation of detergent-range olefinic hydrocarbons from a feed stream comprising one or more olefinic hydrocarbons and other hydrocarbon species, comprising contacting the feed stream with a bed of adsorbent comprising Type X zeolite under conditions which cause the selective retention of the detergent-range olefinic hydrocarbons on the adsorbent and recovering the retained detergent-range olefinic hydrocarbons from the adsorbent by contacting the adsorbent with a desorbent comprising one or more naphthenic hydrocarbons.

18. The process of claim 17 wherein the detergent-range olefinic hydrocarbons comprise olefins within the range of C₉ to C₂₀.

19. The process of claim 18 wherein the detergent-range olefinic hydrocarbons consist essentially of olefins within the range of C₉ to C₂₀.

20. The process of claim 17 wherein the naphthenic hydrocarbons consists essentially of one or both of cyclohexane and methylcyclopentane.
